(19) United States
(12) Patent Application Publication
(10) Pub. No.: US 2011/0282046 A1
(43) Pub. Date: Nov. 17, 2011

(54) PROCESS FOR PREPARATION OF CIS-NUCLEOSIDE DERIVATIVE

(76) Inventors: Rama Shankar, Andhra Pradesh (IN); Ravinder Raddy Vennapureddy, Andhra Pradesh (IN); Asif Parvez Sayyed, Andhra Pradesh (IN); Murali Krishna Ankaraju, Andhra Pradesh (IN); Suri Babu Madasu, Andhra Pradesh (IN); Janardhan Rao Vascuri, Andhra Pradesh (IN); Sivakumaran Meenakshisunderam, Andhra Pradesh (IN)

(21) Appl. No.: 13/138,204

(22) PCT Filed: Jan. 15, 2010
(86) PCT No.: PCT/IB2010/000078

§ 371 Date: Jul. 19, 2011

(30) Foreign Application Priority Data
Jan. 19, 2009 (IN) 118/CHE/2009
May 28, 2009 (IN) 1237/CHE/2009

(51) Int. Cl. C07H 19/06

(52) U.S. Cl. 536/28.5

(57) ABSTRACT

The present invention relates to a novel and stereoselective synthetic process for the preparation of optically active cis-nucleoside derivatives of compound of Formula (I), wherein R₃ represents H, F, Cl, C₁₋₁₆ alkyl.

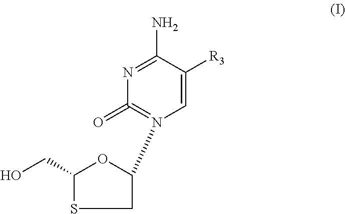

### FIELD OF THE INVENTION

The present invention relates to a novel and stereoselective synthetic process for the preparation of optically active cis-nucleoside derivatives of Formula I,

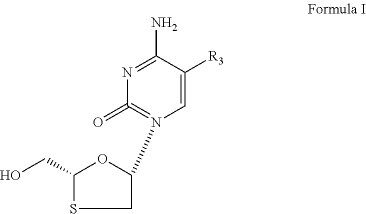

wherein R₃ represents H, F, Cl, C₁₋₁₆, alkyl.
including but not limited to stereoselective preparation of Lamivudine, Emtricitabine and related compounds.

### BACKGROUND OF INVENTION

Cis-Nucleoside derivatives Lamivudine (3TC) and Emtricitabine (FTC) are useful in the treatment of retroviral infections caused by Human immuno deficiency virus (HIV), Hepatitis B virus (HBV) and Human T-Lymotropic virus (HTLV).

Lamivudine (3TC) is presently marketed by GlaxoSmithkline and is available as “EPIVIR” and is disclosed first in U.S. Pat. No. 5,047,407. Emtricitabine is developed by Emory University and marketed by Gilead Sciences Inc., in the name of EMTRIVA and TRUVUDA and is first disclosed in U.S. Pat. No. 5,814,639.

The oxathiolane derivatives, such as Lamivudine (3TC) of Formula-II and Emtricitabine (FTC) of Formula-III,

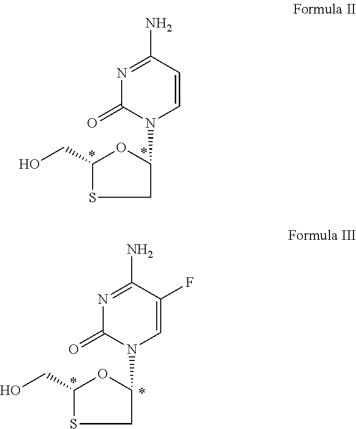

have two chiral centres and it can have four stereo isomers namely (2R,5S), (2S,5S), (2R,5R) and (2S,5R). The Pharmaceutically more active and less cytotoxic isomer is (−)-cis-isomer and has an absolute configuration (2R,5S) in both Lamivudine and Emtricitabine.

U.S. Pat. No. 5,047,407 and U.S. Pat. No. 6,903,224 B2 disclose the preparation of Lamivudine as a racemic mixture i.e. (±) 3TC. The process comprises, reacting silylated cytosine with 2-benzoyloxymethyl-5-ethoxy-1,3-oxathiolane in presence of trimethylsilyl triflate (TMSTf) for three days under reflux to yield glycosidated product. Glycosidated product was isolated after chromatography as a mixture of cis and trans isomers (1:1). The isomeric mixture obtained after chromatography was acetylated and the acetylated mass was again subjected to chromatography for separating cis and trans isomers. Thereafter the diprotected cis or trans derivative was deprotected in basic medium (methanolic ammonia) to yield racemic Lamivudine. As evident, this is not the industrially desired condition where the glycosidation reaction is carried out for three days at reflux temperature, the reaction results in isomeric mixture (1:1) and the reaction product requires extra acetylation step for isomeric separation, which involves repeated chromatographic separation and as a result of all these gives a very low overall yield of the racemic product.

U.S. Pat. No. 6,831,174 and U.S. Pat. No. 6,175,008 disclose a process to prepare racemic Lamivudine wherein the glycosidation reaction of silylated pyrimidine bases such as uracil /cytosine derivative and the 1,3-oxathiolane moiety, was carried out in the presence of Lewis acids such as TiCl₄, ZnCl₂, TMSI, TMSTf, SnCl₄ or a mixture of ZnCl₂ and TiCl₄. Glycosidation using all these Lewis acids has resulted in the formation of mixture of cis and trans isomer in different proportions with almost 50:50 in few cases. Further, an additional step of acylation was required to separate the cis and trans isomer. The mixture or individual isomer yield was between 31-60%. TMSI had the best selectivity among all the Lewis acids. In U.S. Pat. No. 6,831,174, it is stated that when halogen containing silyl Lewis acid such as trimethyl silyl iodide is used, it converts the existing leaving group into iodo and results in better selectivity for the cis-isomer.

U.S. Pat. No. 6,153,751 and U.S. Pat. No. 5,248,776 disclose a process to prepare)-3-isomer of 1,3-oxathiolane pyrimidine nucleoside as racemic mixture by reacting 5-(O-protected)-2-(protected hydroxymethyl)-1,3-oxathiolane derivative with silylated pyrimidine base in the presence of SnCl₄. Also, the enantiomerically enriched 3TC and its analogs have been prepared by using the chirally pure 5-(O-protected)-2-(protected oxymethyl)-1,3-oxathiolane. The chirally pure intermediate was prepared either by enzymatic resolution, where 50% of the undesired isomer is lost resulting in lower overall efficiency of the synthesis or by low yielding multi step tedious synthesis starting from chiral sugar e.g. L-gulose. In addition to the difficulty in handling the highly air and moisture sensitive, corrosive, fuming SnCl₄, use of SnCl₄ in nucleoside synthesis are known to pose problems due to formation of undesirable emulsions during the work-up of the reaction mixture. Some times it generates inseparable complex mixtures of α and β isomers, which requires commercially unviable, repeated column separations. Further, it may also result in the formation of a number of stable δ-complexes between the SnCl₄ and the basic silylated heterocycles, which are difficult to remove and thus results in lower yield.

Most of the above described approach of synthesis results in racemic mixtures. In U.S. Pat. No. 6,180,639, U.S. Pat. No. 5,728,575, U.S. Pat. No. 7,160,999, U.S. Pat. No. 6,703,396 the optical active isomer of (−)-3TC or (−)-FTC has been obtained from the racemic mixture either by preparative HPLC using chiral column or by enzymatic separation involving the enantioselective hydrolysis of 5′-nucleoside or by the enzyme assisted selective deamination reaction.

U.S. Pat. No. 6,051,709 and U.S. Pat. No. 5,696,254 describes a stereocontrolled synthesis of the desired cis-nucleoside analogue starting from optically pure intermediate. As per the process disclosed in U.S. Pat. No. 5,696,254, the glycosidation reaction is carried out in the presence of a silylated Lewis acid R₅R₆R₇R₈Si e.g. iodotrimethyl silane. The process is as shown below:

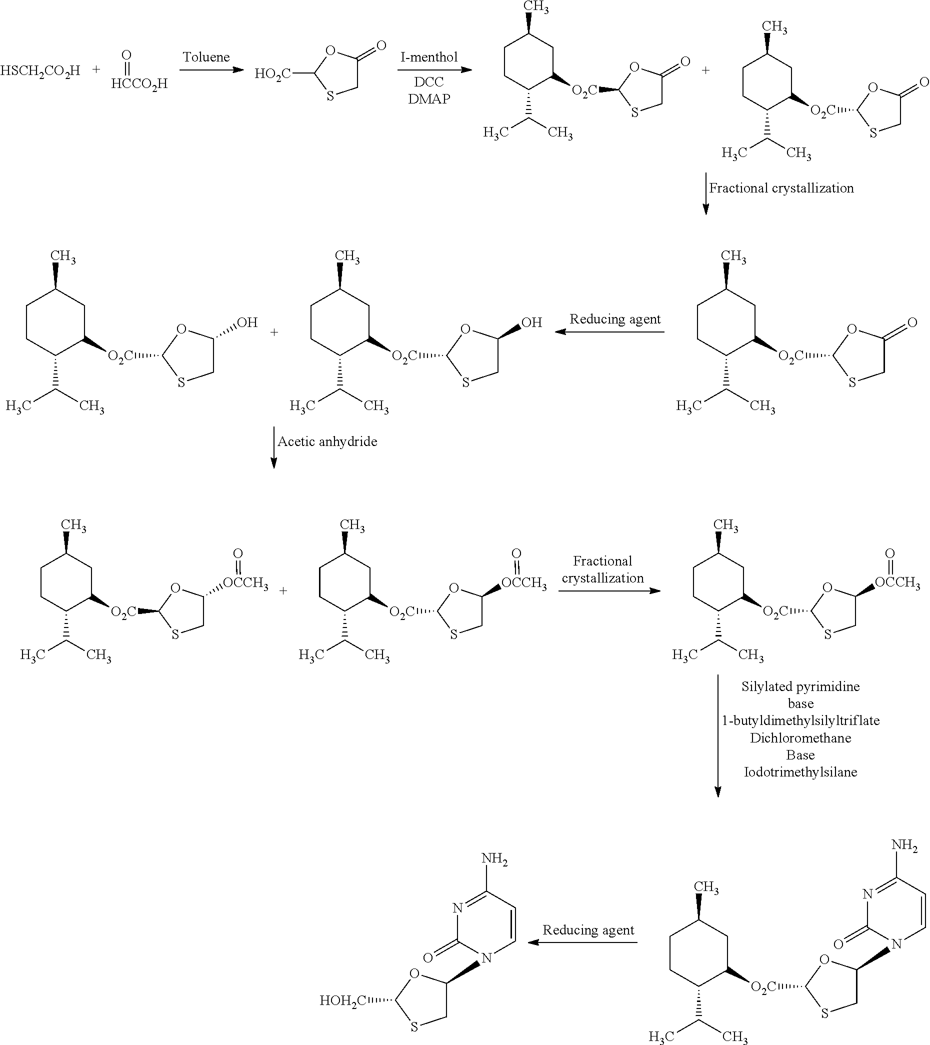

As per the process disclosed in U.S. Pat. No. 6,051,709 the glycosidation is effected without the Lewis acid catalyst, however the leaving group is limited to halo, cyano or R₉SO₂. The process is as shown below:

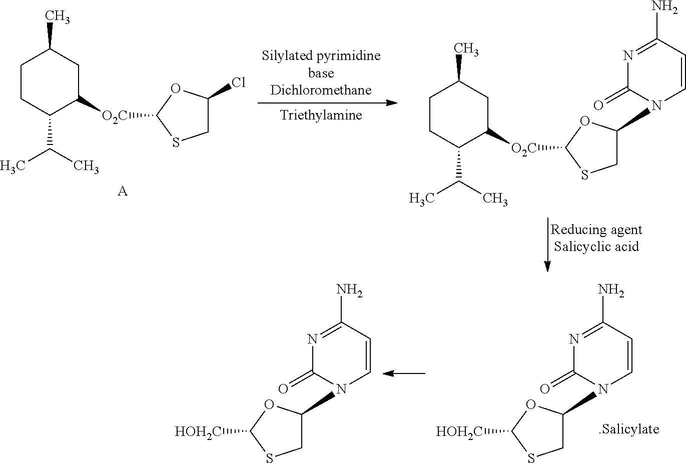

There are number of disadvantages associated with the use of silylated Lewis acid as they are highly reactive, moisture sensitive and unstable compounds. They are very expensive and have significant toxic effects. In U.S. Pat. No. 6,051,709 for preparing intermediate A, halogenating agent was used, which is selected from oxalyl halide, thionylhalide, phosphoroushalide, phosphorousoxyhalide. These are corrosive, hazardous and moisture sensitive compounds. In addition, hydrogen halide byproduct generated during the preparation of the halo leaving group may also result in destruction/decomposition of acid sensitive 1,3-oxathiolane moiety.

U.S. Pat. No. 6,939,965 discloses the glycosidation of silylated 5-fluorocytosine with oxathiolane having a protected hydroxylmethyl group at second position of the oxathiolane ring. The glycosidation reaction is carried out using a Lewis acid, TiCl₃(OiPr) to give the desired (2R,5S) isomer in excess and it is further purified by fractional crystallization.

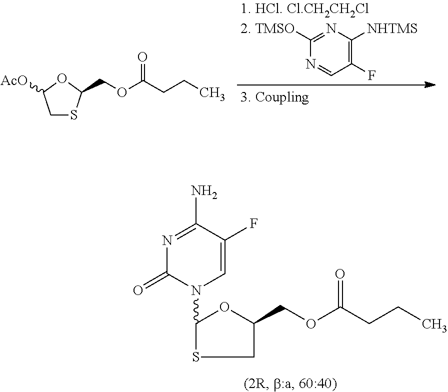

WO 2004/085432 A1 discloses a process to prepare emtricitabine, by condensing 5-fluorocytosine with activated 1,3-oxathiolane in the presence of a Lewis acid to give an intermediate compound. This intermediate compound was dissolved in a solvent and treated with organic or mineral acids selected from oxalic acid, succinic acid, maleic acid, methanesulphonic acid, 4-chlorobenzenesulphonic acid, hydrochloric acid, to give an intermediate salt. Thereafter, the intermediate salt was desalified in situ and reduced using a reducing agent to give emtricitabine.

Therefore, there is currently a need for a general and economically attractive, efficient stereoselective synthesis of biologically active cis-nucleoside analogue such as Lamivudine, Emtricitabine where enantiomerically enriched β-isomer could be obtained using inexpensive reagents/catalysts in good yield with minimum formation of the undesired α-isomer.

In the course of our investigation on the development of stereoselective glycosidation reaction for the efficient synthesis of 3TC, FTC and its analogues surprisingly it was discovered that triphenylmethyl perchlorate (trityl perchlorate) is an effective reagent for the glycosidation reaction. This reagent has been previously used for the synthesis of C-nucleoside/C-ribofuranoside (Chemistry Letters 1984, 907, 1529), where it was used during reaction of sugars with alcohols for the activation of acyloxy group on the anomeric centre of 1-O-acyl sugars. However, it has not been explored for the synthesis of 3TC, FTC and their analogues having a 1,3-oxathiolane moiety. The term analogues is meant to refer to nucleosides that are formed from pyrimidine bases substituted at the 5^{th}. position that are coupled to substituted 1,3-oxathiolane moiety.

### OBJECTIVE

The main objective of the present invention is to provide an improved stereoselective process for preparing 1,3-oxathiolane nucleoside and its intermediate, in cis-configuration, which over comes the disadvantages of prior art processes.

Yet another objective of the present invention is to provide an improved process to obtain optically pure intermediates useful in preparation of (−)-3TC and (−)-FTC and their analogues by making use of safe reagent/catalyst in glycosidation reaction and isolation of desired stereo isomer from the diastereomeric mixture.

Yet another objective of the present invention is to provide an improved process to prepare 3TC and (−)-FTC and their analogues, which is simple, industrially applicable and economically viable.

### SUMMARY OF THE INVENTION

The present invention relates to isolation of Lamivudine/Emtricitabine as water insoluble succinic acid salt or as cinnamate salt efficiently from aqueous solutions, and further conversion in to Lamivudine/Emtricitabine.

In another embodiment the present invention also relates to an improved process to prepare a cis-nucleoside derivative of Formula I,

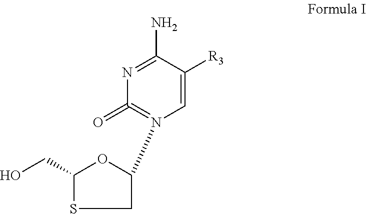

wherein R₃ represents H, F, Cl, C₁₋₁₆ alkyl. which comprises:

a) reacting 1,3-oxathiolane compound of Formula IV,

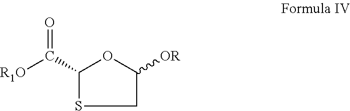

wherein R represents acyl group selected from —COCH₃, —COC2H₅, —COCH₂Cl, —COCH₂Br, —COC₆H₅, —COR_{S}; R₅ represents substituted phenyl group selected from 4-nitrophenyl, 4-chlorophenyl; R₁ represents hydrogen, alkyl, aralkyl, alkenyl, aryl preferably an alkyl group/substituted alkyl group, more preferably a chiral auxiliary with one or more chiral centres such as d(+)menthol or l(−)menthol.
with a pyrimidine base of Formula V,

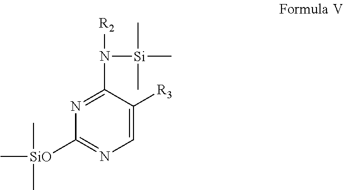

wherein R₂ represents H or COR₄; R₄ represents H, C₁₋₆ alkyl; R₃ represents H, F, Cl, C₁₋₁₆ alkyl.
in the presence of trityl glycosidation agent to give protected nucleoside of major [2R, 5S] compound of Formula VI and minor [2R, 5R] compound of Formula VII;

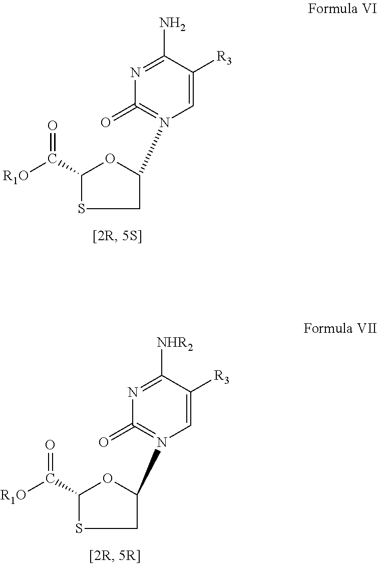

wherein R₁, R₂ and R₃ are same as defined above.
b) optionally isolating the compound of Formula VI;
c) deprotecting the mixture [2R, 5S] compound of Formula VI and [2R, 5R] compound of Formula VII in presence of an acid to give a [2R, 5S] compound of Formula VIII and [2R, 5R] compound of Formula IX;

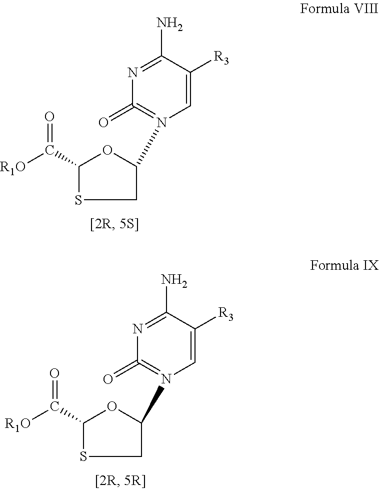

wherein R₁ and R₃ are same as defined above.
d) isolating the [2R, 5S] compound of Formula VIII;
e) reducing the [2R, 5S] cis-nucleoside compound of Formula VIII to give compound of Formula I; and
f) isolating the cis-nucleoside derivative.

In another embodiment of the present invention, the deprotected cis-nucleoside compound of Formula VIII is isolated as a salt, of compound of Formula XVII and compound of Formula XVIII,

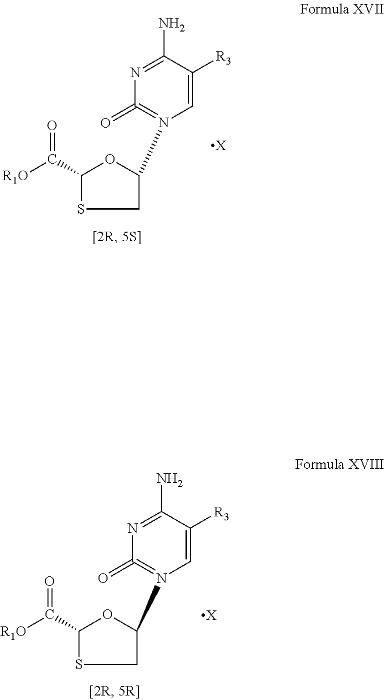

wherein R₁, and R₃ are same as defined above; X represents methanesulphonic acid, trifluoroacetic acid, p-toluenesulfonic acid,
and further converted in to Lamivudine/Emtricitabine.

In another embodiment of the present invention, the Lamivudine can be obtained as polymorphic Form I and polymorphic Form II.

### DETAILED DESCRIPTION OF THE INVENTION

The 5-O-acyl-1,3-oxathiolane derivative of Formula IV is condensed with a silylated pyrimidine base or N-protected silylated pyrimidine base of Formula V, which includes cytosine or N-alkanoyl-cytosine, 5-fluorocytosine or N-alkanoyl-5-fluorocytosine, in the presence of efficient trityl glycosidation agent selected from trityl perchlorate of compound of Formula X,

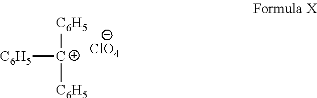

or trityl fluoroborate of compound of Formula XI,

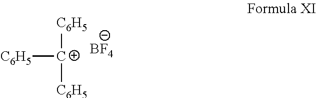

to provide the corresponding nucleoside with high β-selectivity. It will be understood that if the coupling step is carried out using a racemic 1,3-oxathiolane derivative, a racemic mixture of cis-nucleoside analog will be obtained. However, it is preferred that coupling is affected using an optically pure 1,3-oxathiolane compound of Formula IV, thereby producing the desired cis-nucleoside analog in high optical purity.

The coupling reaction of silylated base with oxathiolane derivative is carried out at temperature in the range of 10-80° C., preferably at 40-60° C. in a solvent selected from halogenated hydrocarbons such as methylene chloride, ethylene chloride; hydrocarbon such as toluene; a nitrile such as acetonitrile; an ether such as tetrahydrofuran; 1,2-dimethoxy ethane (DME) or mixtures thereof. The coupling reaction is preferably carried out in methylene chloride or toluene as a solvent, more preferably in a mixture of toluene and methylene chloride. The trityl glycosidation agent can be used in 0.3-1.3 equivalent moles based on the oxathiolane compound. However, better stereoselectivity (β-isomer≧80%) and lower reaction time was achieved when equivalent mole is used. When lower quantity of trityl glycosidation agent (˜0.3 m. eq.) is used it takes longer time to complete the reaction.

When pyrimidine bases with protected amino group such as 4-N-acyl e.g., 4-N-acetyl, 4-N-propionyl etc., 4-N—(N,N-dimethylamino methylene) were silylated and used in the coupling, the rate of reaction was better, with minimum side product formation and higher β-selectivity was obtained. Among the 4-N-acyl protected pyrimidine bases 4-N-propionyl protected base was most preferred as in the glycosidation reaction with 4-N-acetyl cytosine formation of a major side product (˜18% by HPLC analysis, Formula XII) was observed, which decreases the yield of the reaction. Similar side reactions were not observed when instead of N-acetyl cytosine, N-propionyl or N-butanoyl substituted cytosine was used for coupling reactions, this also results in the better yield of the coupled product.

The ratio of β:α isomer during the coupling in methylene chloride and toluene mixture was ˜80:20. However, when only acetonitrile or acetonitrile-toluene mixture was used as a solvent, the formation of desired β-isomer was comparatively lower (β:α isomer ˜71:29). The pure cis N-alkanoyl nucleoside compound of Formula VI could be separated from the reaction mass (containing a diastereomeric mixture, 80% of β i.e. 2R, 5S, 20% of α, i.e. 2R, 5R) directly or a crude having the trans isomer up to 3% is isolated and then subjected to crystallization from a mixture of solvents preferably ethyl acetate and hexanes to get pure cis isomer having trans isomer less than 1%. Further, the isolated N-alkanoyl nucleoside derivative was subjected to deacylation by hydrolysis using an acid preferably in trifluoroacetic acid, methane sulfonic acid or p-toluenesulfonic acid in an alcoholic solvent preferably absolute ethanol. The deacylated nucleoside in the form of acid addition salt could be isolated as such from the reaction mass or by adding an anti solvent such as hydrocarbons selected from toluene, hexanes, cyclohexane or an ether solvent selected from diisopropylether or an ester solvent selected from ethylacetate, isopropylacetate. The deacylated nucleoside could also be isolated as base from reaction mass by adding an organic base such as triethylamine.

In order to minimize the loss in two isolations (isolation of N-alkanoyl nucleoside derivative and isolation of nucleoside derivative), the reaction product having diastereomeric mixture is further subjected to acidic hydrolysis in alcoholic solvent e.g., in ethanol with methane sulfonic acid, trifluoroacetic acid or p-toluenesulfonic acid. Hydrolysis is completed in ˜5-6 h at ambient temperature when methane sulfonic acid is used. However, in case of trifluoroacetic acid warming of reaction mass to ˜45° C. is required. An anti solvent selected from hydrocarbons selected from toluene, hexanes, cyclohexane or an ether solvent selected from diisopropylether or an ester solvent selected from ethylacetate, isopropylacetate is added to precipitate the pure β-isomer as acid addition salt. Contrary to the literature reports where an additional acylation step was carried out to separate the cis and trans isomers, pure cis isomer (having less than 2% trans isomer) could be recovered directly as methane sulfonic acid, p-toluenesulfonic acid or trifluoroacetic acid salt from a reaction mass containing a mixture of two isomers. Therefore, this presents an important advantage over the prior art processes and is an aspect of the instant invention where α and β isomers have been separated easily from the mixture as an acid addition salt. The acid addition salt thus obtained is neutralized with an organic base, preferably triethylamine in a mixture of solvents preferably a mixture of ethyl acetate, hexanes and water. It was also noted that neither during the coupling using the trityl perchlorate catalyst nor during the acidic hydrolysis for acyl deprotection, any reacemization has taken place and the stereointegrity is maintained during the process. Lamivudine coupled ester/Emtricitabine coupled ester compound of Formula VIII prepared as per the above process is having a chromatographic purity of above 99% and chiral purity ˜100% and the diastereomer (2R,5R) of compound of Formula IX content is ≦0.3%.

The compound of Formula VIII is reduced using sodium borohydride to get 3TC or FTC or its analogue. The reduction reaction is normally carried out in aqueous alcoholic solvent, mixture of alcoholic solvent with water or aqueous tetrahydrofuran, thus resulting in a solution of Lamivudine/Emtricitabine, along with the reaction byproducts [e.g. l(−)menthol] and the inorganics in aqueous alcohol. In view of the high solubility of Lamivudine/Emtricitabine in aqueous alcohol, isolation of product from such a solution is difficult by conventional methods. This difficulty has been overcome in U.S. Pat. No. 6,051,709 by preparing specifically the salicylate salt, which are sparingly soluble in water and allows Lamivudine/Emtricitabine to get precipitated from water as salicylate salt. Although, the preparation of salicylate salt allows the isolation of product from water, preparation of salicylate salt has a serious problem as the salicylic acid is almost insoluble in water (0.20 g in 100 ml water, Merck Index Entry no. 8332) and therefore the precipitated salt is also accompanied with the unreacted salicylic acid and thus contaminates the product.

We have found that Lamivudine/Emtricitabine may be efficiently isolated from an aqueous solution as water insoluble succinic acid salt or as cinnamate salt. Succinic acid having good solubility in water (1 g dissolves in 1 ml of boiling water or 13 ml of cold water, Merck Index Entry no. 8869) any succinic acid left unreacted during salt preparation gets eliminated in mother liquor during the filtration of the succinate salt and do not contaminate the product and thus overcomes the major draw back of prior art process and produces significant advantage over salicylate salt preparation. The succinate salt isolated was found to be equimolar salt having a mole of water attached with it. The preparation of water insoluble cinnamate salt affords similar advantage, as any unreacted cinnamic acid can be easily be washed out using hydrocarbon solvents such as toluene, which are usually used in the process to remove the process byproduct l(−)menthol. Another advantage with cinnamate salt is realized when the cinnamate salt of Lamivudine is subsequently converted to the free base in organic solvent using organic tertiary base eg. triethylamine. Because of the solubility of cinnamate salt of organic base in most of the organic solvents, removal of the byproduct in isolation of Lamivudine free from any contaminant becomes very easy. Lamivudine was found to crystallize as dicinnamate salt i.e. for each mole of Lamivudine two moles of cinnamic acid is attached. Contrary to the salicylate/succinate salt the Lamivudine dicinnamate salt always crystallized as anhydrous product (with MC <0.3% w/w) from water solution. Attempt to prepare mono cinnamate salt of Lamivudine from water did not succeed. In fact, when equimolar quantity of cinnamic acid was added only 50% of product had crystallized as dicinnamate salt and the rest of the 50% had gone in aqueous mother liquor as free base.

The succinate or dicinnamate salt may be prepared by treating aqueous solution as such obtained from reduction reaction containing Lamivudine/Emtricitabine with succinic acid/cinnamic acid. A water miscible cosolvent such as methanol, ethanol, dioxane, tetrahydrofuran or mixture of these solvents could be also added during salt preparation. These salts of Lamivudine/Emtricitabine are subsequently converted into free bases in organic solvent by treatment with suitable organic bases selected from tertiary amines. Lamivudine from these salts was obtained as either Lamivudine polymorphic Form-I or Lamivudine polymorphic Form-II based on the solvents and reaction conditions used for such conversion.

In a further aspect of the present invention, Lamivudine polymorphic Form-II is obtained by treating Lamivudine salt in an organic solvent or mixture of organic solvents or in a mixture of organic solvent and water in the presence of a base. The organic solvent is selected from ethyl acetate, methyl acetate, ethanol, methanol, isopropyl alcohol, n-propanol, n-butanol, acetone; the base is selected from triethyl amine, ammonium hydroxide, ammonia, t-butyl amine etc., preferably triethyl amine.

In a further aspect of the present invention, Lamivudine polymorphic Form-II is obtained by treating Lamivudine salt in an organic solvent in the presence of a base. The organic solvent is selected from ethanol, ethyl acetate, isopropyl alcohol; the base is selected from triethyl amine, diethyl amine, diisopropyl amine etc., preferably triethyl amine.

In a further aspect of the present invention, the Lamivudine/Emtricitabine are isolated from aqueous solution as water insoluble sulfoxide. The aqueous solution of Lamivudine along with the inorganic impurities obtained from the reduction reaction was washed with a water immiscible solvent such as toluene/methylene chloride/cyclohexane/hexanes etc., to remove the byproduct menthol. Thereafter, the aqueous solution is treated with an oxidizing agent selected from hydrogen peroxide. It was surprisingly found that the Lamivudine sulfoxide precipitates from water leaving behind the inorganic impurities. Therefore, affords an alternative and efficient method for the isolation of Lamivudine from such media and removal of inorganic impurities. The sulfoxide thus obtained is treated with phosphorous pentasulfide (P₄S₁₀) in organic solvent to carry out deoxygenation and to obtain Lamivudine. The deoxygenation reaction is carried out under mild reaction condition in organic solvents selected from methylene chloride, toluene, acetone, pyridine, carbon disulphide or mixture of these solvents where the reaction byproducts are soluble and Lamivudine is completely insoluble. The deoxygenation reaction is carried out at RT or at ˜45° C. for 10-16 h.

The Lamivudine is isolated as crystalline polymorphic Form I or crystalline polymorphic Form II.

The N-protected pyrimidine base of Formula V is prepared by known literature procedures e.g., N-acyl cytosine such as N-propionyl cytosine.

The N-propionyl cytosine is prepared by reacting cytosine with propionic anhydride in toluene in presence of pyridine base and catalytic amount of dimethylamino pyridine (DMAP). 4-N—(N,N-dimethylamino methylene)cytosine was prepared by reaction of cytosine with N-dimethylformamide dimethylacetal (DMF-dimethylacetal).

Silylated pyrimidine bases of Formula V were prepared as per the literature procedure, by reacting pyrimidine base or N-protected pyrimidine base with a silylating agent such as hexamethyl disilazane (HMDS) with a drop of methane sulfonic acid or a pinch of ammonium sulfate in toluene solvent. The toluene solution containing silylated product was used as such for the coupling reaction.

The 1,3-oxathiolane derivative of Formula IV was prepared by literature procedure e.g. l(−)menthyl glyoxalate hydrate was prepared by reaction of l(−)menthol with glyoxalic acid as described in U.S. Pat. No. 5,489,705, l(−)menthol glyoxalate hydrate is treated with 1,4-dithiane-2,5-diol as per literature procedure described in U.S. Pat. No. 6,051,709 to give (2R,5R)-5-hydroxy-1,3-oxathiolane-2-carboxylic acid l(−)menthyl ester. The 5-hydroxy compound is reacted with an acid anhydride/acid chloride as per the known methods (e.g.: T. W. Greene “Protective groups in organic synthesis, John Wiley & Sons, New York) to get the 5-O-acyl oxathiolane derivative.

A small sample of the major side product ((˜18% by HPLC analysis) formed during the coupling of 1,3-oxathiolane and N-acetyl cytosine) was separated by column chromatography and characterized by mass and ¹H & ¹³C NMR. The ¹H NMR spectrum of the above compound shows a clean singlet at δ 3.98 for the two protons of COCH₂ group and the remaining protons were in the expected region. The mass spectra shows a molecular ion peak at m/z 665 with a m/z 243 (trityl fragment). Based on the characterization data, the structure of the side product was established as compound of Formula XII.

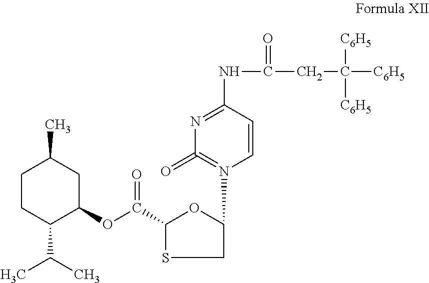

Although, the electron withdrawing nature of amide carbonyl is minimum, probably as the —NH group is attached to an aromatic nucleus, the methyl group becomes acidic enough that a proton can be picked to generate CH₂′. The anion generated can easily react with the stable trityl carbo-cation leading to the formation of compound of Formula XII. This non-polar impurity gets completely eliminated during the isolation of the compound of Formula VI or the corresponding deacetylated product compound of Formula VIII without effecting the quality of the product. Another significant observation was that during the deacetylation of the compound of Formula VI by acidic hydrolysis using methane sulfonic acid /trifluoroacetic acid or p-toluenesulfonic acid in alcohol to get product of Formula VIII, the —COCH₂C(C₆H₅)₃ did not get hydrolyzed.

The present invention also relates to novel compound of Formula XII

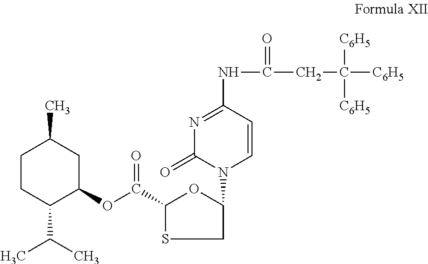

The present invention also relates to novel compound of Formula XIII,

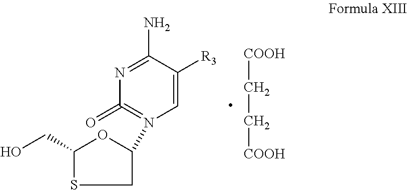

wherein R₃ represents H, F, Cl, C₁₋₁₆ alkyl.

The present invention also relates to novel compound of Formula XIII, which is isolated as a monohydrate.

The present invention also relates to novel compound of Formula XIV,

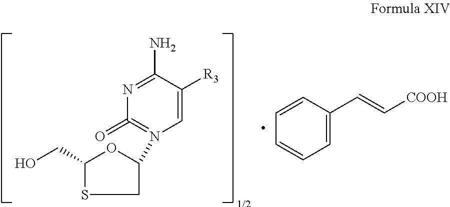

wherein R₃ represents H, F, Cl, C₁₋₁₆ alkyl.

The present invention also relates to novel compound of Formula XV,

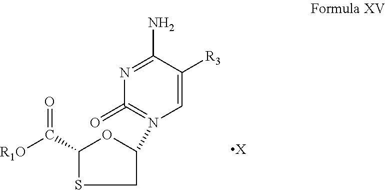

wherein R₁ represents hydrogen, alkyl, aralkyl, alkenyl, aryl preferably an alkyl group/substituted alkyl group, more preferably a chiral auxiliary with one or more chiral centres such as d(+)menthol or l(−)menthol; R₃ represents H, F, Cl, C₁₋₁₆ alkyl, X represents methanesulphonic acid, trifluoroacetic acid, p-toluenesulfonic acid.

The present invention also relates to novel compound of Formula XVI,

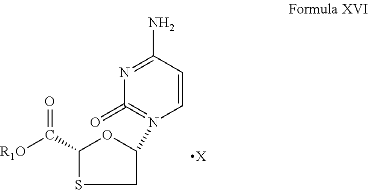

wherein R_{I} represents hydrogen, alkyl, aralkyl, alkenyl, aryl preferably an alkyl group/substituted alkyl group, more preferably a chiral auxiliary with one or more chiral centres such as d(+)menthol or l(−)menthol; X represents methanesulphonic acid, trifluoroacetic acid, p-toluenesulfonic acid.

The invention is illustrated with the following examples, which are provided by way of illustration only and should not be construed to limit the scope of the invention.

### EXAMPLE 1

### Preparation of N-Acetylcytosine

To a suspension of cytosine (200 g, 1.80 mol) in toluene (600 ml) at RT (25-30° C.), pyridine (216 g, 2.73 mol) and DMAP (1.0 g) was added. Acetic anhydride (217 g, 2.13 mol) was diluted with toluene (350 ml) and added slowly in ˜60 min at 25-45° C. After addition, the reaction mass was heated to 50-55° C. and continued stirring for 6 h to complete the reaction (checked by TLC). Reaction mass was cooled to 25-28° C. and product was filtered and washed with toluene (350 ml). Further, product was washed with water (2×300 ml). Product was dried at 60-65° C. under reduced pressure to give title compound.

### Yield: 254 g

¹H NMR (DMSO-d₆): δ 2.08 (s, 3H, CH₃), 7.08-7.10, (d, 1H, CH-cytosine), 7.79-7.81 (d, 1′-1, CH-cytosine), 10.75 (broad singlet, 1H, NH), 11.50 (broad singlet, 1H, NH).

### EXAMPLE 2

### Preparation of N-Propionylcytosine

To a suspension of cytosine (200 g, 1.80 mol) in toluene (700 ml) at RT (25-30° C.), pyridine (222 g, 2.81 mol) and DMAP (1.0 g) was added. The suspension was heated to ˜45° C. and propionic anhydride (273 g, 2.10 mol) was diluted with toluene (500 ml) and added slowly over 2 h at 45-55° C. After completion of addition, stirring was continued at 55-58° C. for 9 h to complete the reaction (checked by TLC). Reaction mass was cooled to 25-28° C. and product was filtered and washed with toluene (350 ml). Further, product was washed with water (2×300 ml). The wet product was stirred for 90 min at 38-40° C. in water (1500 ml) containing triethylamine (6 g). Product was filtered and washed with water (2×300 ml). Product was dried at 50-55° C. under reduced pressure to give title compound.

Yield: 272.3 g.

¹H NMR (DMSO-d₆): δ 0.99-1.04, (1, 31-1, CH₃), 2.35-2.43, (q, 2H, CH₂), 7.11-7.13 (d, 1H, CH-cytosine), 7.79-7.82 (d, 1H, CH-cytosine), 10.71 (broad singlet, 1H, NH), 11.49 (broad singlet, 1H, NH).

### EXAMPLE 3

### Preparation of (1′R,2′S,5′R)-menthyl-5-acetoxy-1,3-Oxathiolane-2R-carboxylate

To a precooled suspension of (1′R,2′S,5′R)-menthyl-5R-hydroxy-1,3-oxathiolane-2R-carboxylate (Hydroxy compound, 300 g, 1.04 mol) in diisopropyl ether (1050 ml) at 0-5° C., pyridine (111.8 g, 1.41 mol) and 4-dimethylaminopyridine (3 g, 0.02 moles) was added under nitrogen. Acetic anhydride (125.6 g, 1.23 mol) was diluted with diisopropyl ether (250 ml) and added to reaction mass at 0-8° C. in ˜2 h. Stirring of reaction mass was continued at 3-8° C. for 6 h to complete the reaction. After completion of reaction (checked by TLC), the reaction mass was heated to RT (20-30° C.) and diluted with diisopropyl ether (250 ml). Thereafter, reaction mass was washed with warm water (3×500 ml, ˜40° C.) to ensure complete removal of pyridine. Organic layer was concentrated under reduced pressure at less than 45° C. to obtain a residue. To the residue hexanes (420 ml) was added and the contents were heated to ˜50° C. and maintained for 15-20 min at same temperature. Thereafter, it was cooled to RT and stirred for ˜30 min. Further, diethylether (25 ml) was added to the slurry at ˜25° C. and stirred for 30 min. The product slurry was cooled to 4-6° C. and stirred for 2 h at the same temperature. Product was filtered and washed with prechilled hexanes (150 ml, 0-5° C.). Product was dried at ˜45° C. under reduced pressure for 6 h to give title compound.

Yield: 259.5 g

¹H NMR (DMSO-d₆): δ 6.69 (d, 1H, H-5), 5.78 (s, 1H, H-2), 4.60-4.66 (m, 1H, CH—O—CO of menthyl), 3.31-3.37 (m, 2H, H-2) 2.04 (s, 3H, O—COCH₃), {(1.87-1.91, 1.62-1.66, 1.39-1.43, 2H+2H+2H=6H of menthyl), (1.02-1.08, 2H of menthyl), (0.86-0.91 & 0.71-0.74 7H+3H=10H of menthyl)}.

### EXAMPLE 4

### (2R,5S)-5-(cytosin-1-yl)-1,3-oxathiolane-2R-carboxylic acid (1′R, 2′S, 5′R)-menthyl ester (Lamivudine Coupled Ester)

Hexamethyl disilazane (65 g, 0.40 mol) was added to a suspension of N-acetylcytosine (61.2 g, 0.40 mol) in toluene (305 ml) at room temperature. Methanesulfonic acid (0.3 g) was added to the above suspension and the contents were heated to reflux (108-113° C.). Reflux was continued for 3 h to obtain a clear solution and to complete the silylation reaction. Thereafter, ˜80 ml toluene was distilled from the reaction mass at atmospheric pressure. The contents were cooled to 30-35° C. under nitrogen atmosphere and methylene chloride (405 ml) was added. Further, (1′R,2′S,5′R)-menthyl-5-acetoxy-1,3-oxathiolane-2R-carboxylate (134.7 g, 0.41 mol) was added and the resulting solution was cooled to 10-15° C. Trityl perchlorate (137 g, 0.40 mol) was added at 10-30° C. The resulting solution was stirred for 60-70 min at 20-30° C. and thereafter heated to reflux (43-51° C.). Reflux was continued and progress of reaction was followed by qualitative HPLC analysis, till N-acetylcytosine left unreacted was ≦6% (it took ˜12 h to achieve this). After completion of reaction, the reaction mass was diluted with methylene chloride (150 ml) and the reaction mass was cooled to 20-30° C. The reaction mass was poured into 1.2 Lt of 7.0% w/v aqueous sodium bicarbonate solution under stirring at 20-30° C. Organic layer was separated and concentrated under reduced pressure at ˜50° C. to get a residue containing the N-acetyl coupled ester along with its diastereomer (β:α=79:21, by HPLC analysis).

The above residue was dissolved in ethanol (400 ml) and methane sulfonic acid (68 g) was added at 20-30° C. The solution was stirred at 20-30° C., where a precipitation was observed after ˜90 min of stirring. The progress of reaction was followed by qualitative HPLC analysis and it was continued till the starting material N-acetyl coupled ester was ≦1% (it took ˜4 h). The reaction mass was diluted by slow addition of diisopropyl ether (700 ml) and further stirring was continued at 20-30° C. for 2 h to complete the crystallization. The product was filtered and washed with a mixture of ethanol (50 ml) and diisopropyl ether (90 ml). Further, it was washed with diisopropyl ether (80 ml) and sucked thoroughly under nitrogen to remove most of the mother liquor. The Lamivudine coupled ester methane sulfonic acid salt (119 g, wet) was obtained.

Small sample from above wet product was dried and analyzed by ¹H NMR. DMSO-d₆: δ 2.32 (S, 3H, methane sulfonic), 3.38 (dd, 1H, C-4), 3.65 (dd, 1H, C-4), 4.65 (sextet, 1H, OCH menthyl), 5.82 (s, 1H, C-2), 6.13 (d, 7.8 Hz, 1H, C-5″), 6.31 (1, 1H, C-5), 8.26 (d, J=7.8 Hz, 1H, C-6″), 9.61 (d, 2H, NH₂). Chromatographic purity (by HPLC): 98.89%, Diastereoisomer (trans-isomer): 0.76%, Cytosine: 0.21% and other impurity 0.14%. Enantiomeric purity (by HPLC): 100%

The Lamivudine coupled ester methane sulfonic acid salt (119 g, wet, as obtained above) was added to a mixture of ethyl acetate (225 ml) and hexanes (95 ml) at 20-30° C. to obtain a slurry. Triethylamine (24.5 g) was diluted with hexanes (25 ml) and slowly in 15-20 min added to the above slurry at 20-30° C. Stirring was continued for 15-20 min and thereafter water (250 ml) was added. Further, stirring was continued for 1 h. The product was filtered and washed with water (2×75 ml) followed by with a mixture of ethyl acetate (50 ml) and hexanes (30 ml). Product was dried under reduced pressure at 55-60° C. to give (2R,5S)-5-(Cytosin-1-yl)-1,3-oxathiolane-2R-carboxylic acid (1′R,2′S,5′R)-menthyl ester (Lamivudine coupled ester)

Yield: 78.6 g

SOR[α]²⁵_{D}:−115 (c=1 in CH₃OH)

Chromatographic purity (by HPLC): 99.67%, Diastereoisomer (trans-isomer): 0.33% and Cytosine: Nil

Enantiomeric purity (by HPLC): 100%.

### EXAMPLE 5

### Preparation of (1′R,2′S,5′R)-menthyl-5(RS)-acetoxy-1,3-oxathio-lane-2R-carboxylate

To a precooled suspension of (1′R,2′S,5R)-menthyl-5R-hydroxy-1,3-oxathiolane-2R-carboxylate (Hydroxy compound, 300 g, 1.04 mol) in diisopropyl ether (1050 ml) at 0-5° C., pyridine (103.5 g, 1.31 mol) and 4-dimethylaminopyridine (0.35 g, 3 mmoles) was added under nitrogen. Acetic anhydride (123.6 g, 1.21 mol) was diluted with diisopropyl ether (380 ml) and added to reaction mass at 0-8° C. in ˜2 h. Stirring of reaction mass was continued at 3-8° C. for 10 h to complete the reaction. After completion of reaction (checked by TLC), the reaction mass was heated to RT (20-30° C.) and diluted with diisopropyl ether (350 ml). Thereafter, reaction mass was washed with 5% v/v aqueous acetic acid (2×400 ml) followed by with warm water (2×500 ml, ˜40° C.) to ensure complete removal of pyridine. Organic layer was concentrated under reduced pressure at less than 45° C. to obtain a solid residue. To the residue hexanes (390 ml) was added and the contents were heated to ˜50° C. and maintained for 15-20 min at same temperature. Thereafter, it was cooled to RT and stirred for ˜30 min. Further, the product slurry was cooled to −6° C. to −10° C. and stirred for 3 h at the same temperature. Product was filtered and washed with prechilled hexanes (150 ml, −6° C. to −8° C.). Product was dried at ˜45° C. under reduced pressure for 6 h to give title compound.

Yield: 279.6 g

Chromatographic purity (By HPLC, RI-Detector): 99.87%

The product obtained was mixture of (2R,5R) and (2R,5S) isomers with (2R,5R) being the predominant (≧90%) and (2R,5S) being the minor component (≦10%) [by HPLC analysis, RI-detector]. It has been observed during coupled ester preparation that isomer ratio of acetoxy compound has no effect on the ratio of α:β isomers formation in glycosidation reaction.

### EXAMPLE 6

### (2R,5S)-5-(cytosin-1-yl)-1,3-oxathiolane-2R-carboxylic acid (1′R, 2′S, 5′R)-menthyl ester (Lamivudine Coupled Ester)

N-Propionylcytosine (100.2 g, 0.60 mol) was suspended in toluene (370 ml) at room temperature and methanesulfonic acid (0.4 g) was added to the suspension. Hexamethyl disilazane (106.3 g on 100% basis, 0.66 mol) was added to the suspension and the contents were heated to reflux (108-113° C.). Reflux was continued for 4 h to complete the silylation reaction and to obtain a clear solution. Thereafter, ˜160 ml toluene was distilled from the reaction mass at 107-113° C. under atmospheric pressure. The contents were cooled to 40-45° C. under nitrogen atmosphere and a mixture of toluene (195 ml) and methylene chloride (570 ml) was added. Further, (1′R,2′S, 5′R)-menthyl-5(RS)-acetoxy-1,3-oxathiolane-2R-carboxylate (208 g, 0.63 mol) was added and the resulting solution was cooled to 10-15° C. Thereafter, trityl perchlorate (213 g, 0.62 mol) was added in single lot, whereby the reaction mass temperature raised to ˜32° C. due to reaction exotherm. The resulting solution was stirred for 60-70 min at 25-30° C. and thereafter heated to reflux (˜53° C.). Refluxing of reaction mass at 52-55° C. was continued and progress of the reaction was followed by qualitative HPLC analysis. Refluxing was continued till N-propionylcytosine left unreacted was <6% (it took ˜12 h to achieve this). After completion of reaction, the reaction mass was diluted with methylene chloride (480 ml) and the reaction mass was cooled to 20-30° C. The reaction mass was poured into 2.7 Lt of 5.0% w/v aqueous sodium bicarbonate solution under stirring at 20-30° C. Organic layer was separated and aqueous layer was extracted with methylene chloride and combined with the organic layer. The combined organic extract is washed with 1% w/v aqueous sodium bicarbonate solution (700 ml). The solution was concentrated under reduced pressure at ˜50° C. to get a residue containing the N-propionyl coupled ester along with its diastereomer (β:α=81:19, by HPLC analysis). Further, ˜100 ml ethanol was added to the residue and distilled completely under reduced pressure to remove residual methylene chloride/toluene.

The above residue was taken in ethanol (600 ml) and methane sulfonic acid (110 g) was added at 20-30° C. The solution was stirred at 20-30° C., where precipitation was observed after ˜2 h of stirring. The progress of reaction was followed by qualitative HPLC analysis and reaction was continued till the starting material N-propionyl coupled ester was ≦1% (it took ˜5 h). The reaction mass was diluted by slow addition of diisopropyl ether (1030 ml) and stirring was continued at 20-30° C. for ˜90 min to complete the crystallization. The product slurry was cooled to 10-12° C. and filtered. Product cake was washed with a mixture of ethanol (120 ml) and diisopropyl ether (345 ml) and sucked thoroughly under nitrogen to remove most of the mother liquor. The Lamivudine coupled ester methane sulfonic acid salt (206 g, wet) was obtained. It contained diastereoisomer (trans-isomer): 3.34% and Cytosine: 1.34% (by qualitative HPLC analysis). Enantiomeric purity (by HPLC): 100%

The Lamivudine coupled ester methane sulfonic acid salt (206 g, wet, as obtained above) was added to a mixture of ethyl acetate (330 ml) and hexanes (80 ml) at 20-30° C. to obtain slurry. Triethylamine (38.8 g) was diluted with hexanes (65 ml) and slowly in 15-20 min added to the above slurry at 20-30° C. Stirring was continued for 15-20 min and thereafter water (500 ml) was added. Further, stirring was continued for 1 h. The product was filtered and washed with water (2×100 ml) followed by with a mixture of ethyl acetate (105 ml) and hexanes (60 ml). Product was dried under reduced pressure at 55-60° C. to give (2R,5S)-5-(Cytosin-1-yl)-1,3-oxathiolane-2R-carboxylic acid (1′R,2′S,5′R)-menthyl ester (Lamivudine coupled ester).

Yield: 116.3 g

SOR[α]²⁵_{D}: −116.9 (c=1 in C₁₋₁₃OH, on anhydrous basis)

Chromatographic purity (by HPLC): 99.25%, Diastereoisomer (trans-isomer): 0.36%, Cytosine: 0.11% and Coupled ester (Ethyl analogue): 0.28%.

Enantiomeric purity (by HPLC): 99.96%.

### EXAMPLE 7

### (2R,5S)-5-(cytosin-1-yl)-1,3-oxathiolane-2R-carboxylic acid (1′R, 2′S, 5′R)-menthyl ester (Lamivudine Coupled Ester)

N-Propionylcytosine (55 g, 0.33 mol) was suspended in toluene (270 ml) at room temperature and methanesulfonic acid (0.3 g) was added to the suspension. Hexamethyl disilazane (56.6 g, 0.35 mol) was added to the suspension and the contents were heated to reflux (108-113° C.). Reflux was continued for 4 h to complete the silylation reaction and to obtain a clear solution. Thereafter, ˜90 ml toluene was distilled from the reaction mass under atmospheric pressure. The contents were cooled to 30-35° C. under nitrogen atmosphere and a mixture of toluene (100 ml) and methylene chloride (350 ml) was added. Further, (1′R, 2′S, 5′R)-menthyl-5-acetoxy-1,3-oxathiolane-2R-carboxylate (110 g, 0.33 mol) was added and the resulting solution was stirred for ˜10 min at RT. Thereafter, the solution was cooled to 10-15° C. and trityl perchlorate (113 g, 0.33 mol) was added in single lot, whereby the reaction mass temperature raised to ˜32° C. due to exothermic. The resulting solution was stirred for 60-70 min at 25-30° C. and thereafter heated to reflux (˜53° C.). Refluxing of reaction mass at 52-55° C. was continued and progress of the reaction was followed by qualitative HPLC analysis. Refluxing was continued till N-propionylcytosine left unreacted was ≦6% (it took ˜12 h to achieve this). After completion of reaction, the reaction mass was diluted with methylene chloride (250 ml) and the reaction mass was cooled to 20-30° C. The reaction mass was poured into 1.2 Lt of 7.0% w/v aqueous sodium bicarbonate solution under stirring at 20-30° C. Organic layer was separated and further washed with DM water (300 ml). The solution was concentrated under reduced pressure at ˜50° C. to get a residue containing the N-propionyl coupled ester along with its diastereomer (β:α=81:19, by HPLC analysis).

The above residue was dissolved in ethanol (320 ml) and methane sulfonic acid (60.2 g) was added at 20-30° C. The solution was stirred at 20-30° C., where precipitation was observed after ˜2 h of stirring. The progress of reaction was followed by qualitative HPLC analysis and reaction was continued till the starting material N-propionyl coupled ester was ≦1% (it took ˜5 h). The reaction mass was diluted by slow addition of diisopropyl ether (530 ml) and stirring was continued at 20-30° C. for ˜90 min to complete the crystallization. The product was filtered and washed with a mixture of ethanol (60 ml) and diisopropyl ether (150 ml). Further, it was washed with diisopropyl ether (100 ml) and sucked thoroughly under nitrogen to remove most of the mother liquor. The Lamivudine coupled ester methane sulfonic acid salt (116 g, wet) was obtained.

Chromatographic purity (by HPLC): 99.13%, Diastereoisomer (trans-isomer): 0.64% and Cytosine: 0.23%. Enantiomeric purity (by HPLC): 100%

The Lamivudine coupled ester methane sulfonic acid salt (116 g, wet, as obtained above) was added to a mixture of ethyl acetate (200 ml) and hexanes (65 ml) at 20-30° C. to obtain slurry. Triethylamine (24 g) was diluted with hexanes (25 ml) and slowly in 15-20 min added to the above slurry at 20-30° C. Stirring was continued for 15-20 min and thereafter water (300 ml) was added. Further, stirring was continued for 1 h. The product was filtered and washed with water (2×75 ml) followed by with a mixture of ethyl acetate (50 ml) and hexanes (25 ml). Product was dried under reduced pressure at 55-60° C. to give (2R,5S)-5-(Cytosin-1-yl)-1,3-oxathiolane-2R-carboxylic acid (1′R,2′S,5′R)-menthyl ester (Lamivudine coupled ester).

Yield: 73.9 g

SOR[α]²⁵_{D}: −114.7 (c=1 in CH₃OH)

Chromatographic purity (by HPLC): 99.78%, Diastereoisomer (trans-isomer): 0.14% and Cytosine: 0.08%

Enantiomeric purity (by HPLC): 100%.

### EXAMPLE 8

### (2R,5S)-5-(N-4-acetylcytosin-1-yl)-1,3-oxathiolane-2R-carboxylic acid (1′R,2′S,5′R)-menthyl ester(N-acetyllamivudine Coupled Ester)

Hexamethyl disilazane (43 g, 0.27 mol) was added to a suspension of N-acetylcytosine (40 g, 0.26 mol) in toluene (200 ml) at room temperature. Methanesulfonic acid (0.2 g) was added to the above suspension and the contents were heated to reflux (108-113° C.). Reflux was continued for 3 h to obtain a clear solution and to complete the silylation reaction. Thereafter, ˜50 ml toluene was distilled from the reaction mass at atmospheric pressure. The contents were cooled to 30-35° C. under nitrogen atmosphere and methylene chloride (250 ml) was added. Further, (1′R,2′S,5′R)-Menthyl-5-acetoxy-1,3-oxathiolane-2R-carboxylate (88 g, 0.27 mol) was added and the resulting solution was cooled to 10-15° C. Trityl perchlorate (89.5 g, 0.26 mol) was added at 10-30° C. The resulting solution was stirred for 60-70 min at 20-30° C. and thereafter heated to reflux (43-51° C.). Reflux was continued and progress of reaction was followed by qualitative HPLC analysis, till N-acetylcytosine left unreacted was ≦8% (it took ˜12 h to achieve this). After completion of reaction, the reaction mass was diluted with methylene chloride (100 ml) and the reaction mass was cooled to 20-30° C. The reaction mass was poured into 1000 ml of 6.0% w/v aqueous sodium bicarbonate solution under stirring at 20-30° C. Organic layer was separated and aqueous layer along with the emulsions was reextracted with methylene chloride (75 ml). The organic extracts were combined and washed with water (200 ml). The washed organic extract was concentrated under reduced pressure at ˜50° C. to get a residue. Cyclohexane (650 ml) was added to the residue and the contents were refluxed for 30-40 min. The product slurry was cooled to ˜23° C. and stirred for about 30 min. Product was filtered and washed with cyclohexane (75 ml). Further, the wet product was suspended in a mixture of cyclohexane (250 ml) and ethyl acetate (75 ml). The suspension was refluxed for about 25 min and thereafter, the product slurry was cooled to about 23° C. and stirred for about 30 min. Product was filtered and washed with chilled ethyl acetate (50 ml). Product was dried under reduced pressure at ˜50° C. to obtain of N-Acetyllamivudine coupled ester.

Yield: 51.6 g

Chromatographic purity (by HPLC): 97.31%

Diastereoisomer (trans-isomer): 1.92%, Cytosine: 0.28% and other impurities 0.49%

SOR [α]²⁵_{D}: −174.4 (c=1 in CHCl₃, on anhydrous basis).

### Example 9

### (2R,5S)-5-(N-4-propionylcytosin-1-yl)-1,3-oxathiolane-2R-carboxylic acid (1′R,2′S,5′R)-menthyl ester(N-propionyllamivudine coupled ester)

Hexamethyl disilazane (35.4 g, 0.22 mol) was added to a suspension of N-propionylcytosine (33.4 g, 0.20 mol) in toluene (180 ml) at room temperature. Methanesulfonic acid (0.2 g) was added to the above suspension and the contents were heated to reflux (108-113° C.). Reflux was continued for 4 h to obtain a clear solution and to complete the silylation reaction. Thereafter, ˜50 ml toluene was distilled from the reaction mass at atmospheric pressure. The contents were cooled to ˜40° C. under nitrogen atmosphere, then a mixture of toluene (60 ml) and methylene chloride (180 ml) was added. Further, (1′R,2′S,5′R)-menthyl-5-acetoxy-1,3-oxathiolane-2R-carboxylate (70 g, 0.21 mol) was added and the resulting solution was cooled to 10-15° C. Trityl perchlorate (70 g, 0.20 mol) was added at 10-30° C. The resulting solution was stirred for 60-70 min at 20-30° C. and thereafter heated to 53-56° C. Stirring at 55-56° C. was continued and progress of reaction was followed by qualitative HPLC analysis, till N-propionylcytosine left unreacted was ≦8% (it took ˜15 h to achieve this). After completion of reaction, the reaction mass was diluted with methylene chloride (100 ml) and the reaction mass was cooled to 20-30° C. The reaction mass was poured into 800 ml of 6.0% w/v aqueous sodium bicarbonate solution under stirring at 20-30° C. The organic layer was separated and washed with water (200 ml). The washed organic layer was concentrated under reduced pressure at ˜50° C. to get a residue. A mixture of cyclohexane (270 ml) and ethyl acetate (85 ml) was added to the residue and the contents were refluxed for 30-40 min. The product slurry was cooled to ˜18° C. and stirred for about 30 min at this temperature. Product was filtered and washed with chilled ethyl acetate (50 ml). Product was dried under reduced pressure at ˜45° C. to obtain N-Propionyllamivudine coupled ester.

Yield: 46.8 g

SOR [α]²⁵_{D}: −158.4 (c=1 in CHCl₃, on anhydrous basis).

### EXAMPLE 10

### 4-amino-1-[(2R,5S)-2-(hydroxymethyl-1,3-oxathiolan-5-yl)-(1H)-pyrimidin-2-one dicinnamate (lamivudine dicinnamate)

Dipotassium hydrogen phosphate (129 g) was dissolved in DM water (190 ml) at 23-35° C. Ethanol (1000 ml) was added to the above solution and cooled to 18-25° C. (2R,55)-5-(cytosin-1-yl)-1,3-oxathiolane-2R-carboxylic acid (1′R, 2′S, 5′R)menthyl ester (Lamivudine coupled ester; 145 g, 0.38 moles) was added to the above biphasic mixture followed by methanol (75 ml) at 18-25° C. Sodium borohydride (32 g, 0.85 moles) was dissolved in precooled sodium hydroxide solution (0.02% w/v, 190 ml). This sodium borohydride solution was added to the reaction mass over a period of 90 min at 18-25° C. Stirring was continued at 18-25° C. for ˜3 h to complete the reaction. Reaction mass was settled for 1 h and the aqueous (buffer layer) was separated. The aqueous layer was reextracted with ethanol (100 ml). The organic layers were combined and pH was adjusted to ˜5.8 with dilute hydrochloric acid (˜10% w/w, ˜20 ml) to decompose the unreacted sodium borohydride. Thereafter, pH was adjusted back to 7.5-7.8 using 10% w/v aqueous sodium hydroxide solution (22 ml). Most of the ethanol-methanol mixture was removed by distillation at atmospheric pressure (at ˜83° C.). Further, any residual ethanol was distilled under reduced pressure at ˜55° C. The resulting oily residue was dissolved in water (600 ml) and washed with toluene (160 ml) to remove the byproduct (l)-menthol. The aqueous solution was treated with charcoal (5 g) and filtered through a hyflo bed and the bed was washed with water (50 ml) to obtain a clear filtrate (˜760 ml).

Cinnamic acid (107 g, 0.72 moles) was added to the clear filtrate and the suspension was heated at 40-45° C. for 30 min. Thereafter, cooled the product suspension to 20-25° C. and stirred for 3 h to complete the crystallization of the product. Product was filtered and washed with DM water (100 ml) followed by toluene (70 ml). The product was dried under reduced pressure at 40-45° C. to obtain Lamivudine dicinnamate.

Yield: 172 g

¹H NMR (DMSO-d₆): δ 3.05 (dd, 1H), 3.39 (dd, 1H), 3.74 (s, 2H, 5-17 (1, 1H), 5.40 (s, br, 1H), 5.72 (d, 1H), 6.21 (1, 1H), 6.53 (d, j=16 Hz, 2H cinnamic), 7.25 (d, br, 2H), 7.42 (m, 6H), 7.58 (d, j=16 Hz, 2H cinnamic), 7.68 (m, 4H), 7.82 (d, 1H), 12.42 (s, br, 21-1).

### EXAMPLE 11

### 4-amino-1-[(2R,5S)-2-(hydroxymethyl-1,3-oxathiolan-5-yl)-(1H)-pyrimidin-2-one succinate monohydrate (Lamivudine Succinate Monohydrate)

Reduction of Lamivudine coupled ester (145 g) was carried out as described in Example-10 and by similar work-up an aqueous solution (˜760 ml) containing Lamivudine was obtained. Succinic acid (43.7 g, 0.37 mol) was added to the solution and the contents were heated to 35-40° C. for 25-30 min. Thereafter, the product suspension was cooled to 5-8° C. and stirred for 2 h at the same temperature. Product was filtered and washed with prechilled water (100 ml). The product was dried at 40-45° C. under reduced pressure to obtain Lamivudine succinate monohydrate.

Yield: 124.6 g

Moisture content: 5.31% w/w

Succinic acid content (by titrimetry): 32.42% w/w

¹H NMR (DMSO-d₆): δ 2.42 (s, 4H, succinic), 3.03 (dd, 1H), 3.41 (dd, 1H), 3.72 (m, 2H), 5.17 (1, 1H), 5.31 (s, br, 1H), 5.73 (d, 1H), 6.20 (t, 1H), 7.25 (d, br, 2H), 7.82 (d, 1H).

### EXAMPLE 12

### 4-amino-1-[(2R,5S)-2-(hydroxymethyl-1,3-oxathiolan-5-yl)-(1H)-pyrimidin-2-one Succinate monohydrate (Lamivudine Succinate Monohydrate)

Dipotassium hydrogen phosphate (129 g) was dissolved in DM water (190 ml) at 23-35° C. Ethanol (1000 ml) was added to the above solution and cooled to 18-25° C. (2R,5S)-5-(cytosin-1-yl)-1,3-oxathiolane-2R-carboxylic acid (1′R, 2′S, 5′R)menthyl ester (Lamivudine coupled ester; 145 g, 0.38 moles) was added to the above biphasic mixture followed by methanol (75 ml) at 18-25° C. Sodium borohydride (32 g, 0.85 moles) was dissolved in precooled sodium hydroxide solution (0.02% w/v, 190 ml). This precooled sodium borohydride solution was added to the reaction mass over a period of 90 min at 18-25° C. Stirring was continued at 18-25° C. for ˜3 h to complete the reaction (By HPLC analysis Lamivudine coupled ester ≦0.2%). Reaction mass was settled for 1 h and the aqueous (buffer layer) was separated. The aqueous layer was reextracted with ethanol (100 ml). The organic layers were combined and pH was adjusted to ˜5.8 with dilute hydrochloric acid (˜10% w/w, ˜20 ml) to decompose the unreacted sodium borohydride. Thereafter, pH was adjusted back to 7.5-7.8 using 10% w/v aqueous sodium hydroxide solution (22 ml). Most of the ethanol-methanol mixture was removed by distillation at atmospheric pressure (at ˜83° C.). Further, any residual ethanol-methanol was distilled under reduced pressure at ˜55° C. The resulting oily residue was dissolved in water (500 ml) and washed with toluene (160 ml) to remove the byproduct (l)-menthol. The aqueous solution was treated with charcoal (5 g) and filtered through a hyflo bed and the bed was washed with water (80 ml) to obtain a clear filtrate (˜700 ml).

Succinic acid (45 g, 0.38 mol) was added to the clear filtrate and the contents were stirred at 23-28° C. for 6 h to complete the salt formation and product precipitation. Thereafter, the product suspension was cooled to 5-8° C. and stirred for 2 h at the same temperature. Product was filtered under nitrogen atmosphere and washed with prechilled water (80 ml) followed by with chilled acetone (70 ml). The product was dried initially at RT (˜30° C.) and finally at 40-45° C. under reduced pressure to obtain

Lamivudine succinate monohydrate.

Yield: 126.4 g

Moisture content: 5.31% w/w

Succinic acid content (by titrimetry): 32.42% w/w

¹H NMR (DMSO-d₆): δ 2.42 (s, 4H, succinic), 3.03 (dd, 1H), 3.41 (dd, 1H), 3.72 (m, 5.17 (t, 1H), 5.31 (s, br, 1H), 5.73 (d, 1H), 6.20 (1, 1H), 7.25 (d, br, 2H), 7.82 (d, 1H).

### EXAMPLE 13

### 4-amino-1-[(2R,5S)-2-(hydroxymethyl)-3-oxo-1,3-oxathiolan-5-yl]-(1H)-Pyrimidin-2-one (Lamivudine Sulfoxide)

Reduction of Lamivudine coupled ester (145 g) was carried out as described in Example 7 and by similar work-up an aqueous solution (˜700 ml) containing Lamivudine was obtained. Hydrogen peroxide (27 g, ˜48% w/w, ˜0.38 mol) was added slowly in ˜60 min to get the clear solution at 22-30° C. The contents were stirred at 22-30° C. for ˜6 h, where product crystallizes out. Thereafter, the product slurry was cooled to 5-8° C. and stirred for 2 h at the same temperature. Product was filtered and washed with prechilled water (75 ml). The product was dried at 45-50° C. under reduced pressure to obtain Lamivudine sulfoxide.

Yield: 82.5 g

¹H NMR (DMSO-d₆): δ 3.06 (dd, 1H), 3.33 (dd, 1H), 3.72 (m, 1H), 3.78 (m, 1H), 4.64 (t, 1H), 5.48 (t, 1H), 5.80 (d, 1H), 6.72 (dd, 1H), 7.38 (brm, 2H), 7.69 (d, 1H).

### EXAMPLE 14

### 4-amino-1-[(2R,5S)-2-(hydroxymethyl)-1,3-Oxathiolan-5-yl]-(1H)-pyrimidin-2-one (Lamivudine)

Lamivudine sulfoxide (10 g, 41 mmol) was taken in pyridine (40 ml) and phosphorous pentasulfide (4.40 g, 20 mmoles) was added at 22-30° C. under nitrogen atmosphere. Stirring was continued at 22-30° C. for 6 h. Thereafter, the mass temperature was raised to ˜40° C. and further stirred for 4 h at 40-42° C. to complete the reaction, Reaction mass was filtered and the clear filtrate was concentrated under reduced pressure to about half of its volume. The concentrate was diluted with toluene to precipitate the product. Crude product was filtered washed with hot toluene. Further, purification of crude product was carried out by crystallization from ethanol to yield the Lamivudine Polymorphic Form-II.

Yield: 6.3 g.

### EXAMPLE 15

### 4-amino-1-[(2R,5S)-2-(hydroxymethyl-1,3-oxathiolan-5-yl)-(1H)-pyrimidin-2-one (Lamivudine form I)

Lamivudine dicinnamate (100 g, 0.19 moles) was added to ethyl acetate (600 ml) containing water (7 ml) and the resulting slurry was stirred at 22-30° C. for ˜15 min. Triethylamine (44.5 g, 0.44 moles) was diluted with ethyl acetate (80 ml) and added to the above slurry slowly over a period of 30 min at 22-30° C. The product slurry was further stirred at 22-30° C. for 4 hours. The product was filtered and washed with ethyl acetate (2×60 ml). Product was dried under reduced pressure at 40±2° C. to obtain Lamivudine Form I.

Yield: 39.6 g

### EXAMPLE 16

### 4-amino-1-[(2R,5S)-2-(hydroxymethyl-1,3-oxathiolan-5-yl)-(1H)-pyrimidin-2-one (Lamivudine Form II)

Lamivudine dicinnamate (100 g, 0.19 moles) was added to ethanol (350 ml) and the resulting slurry was stirred at 22-30° C. for ˜15 min. Triethylamine (44.5 g, 0.44 moles) was diluted with ethanol (50 ml) and added to the above slurry slowly over a period of 30 min at 22-30° C. The product slurry was further heated to reflux (˜80° C.) and continued till a clear solution was obtained. The solution was cooled to ˜55° C. and seeded with Lamivudine Form-II (0.1 g) and further cooled to 15-18° C. Stirring was continued at 15-18° C. for 2 h. The product was filtered and washed with precooled ethanol (50 ml). Product was dried under reduced pressure at 50-55° C. to obtain Lamivudine Form II.

Yield: 38.2 g

### EXAMPLE 17

### 4-amino-1-[(2R,5S)-2-(hydroxymethyl-1,3-oxathiolan-5-yl)-(1H)-pyrimidin-2-one (Lamivudine Form I)

Lamivudine succinate monohydrate (100 g, 0.27 moles) was added to a mixture of ethanol (210 ml) and water (24 ml) and the resulting slurry was stirred at 22-30° C. for ˜15 min. Triethylamine (57.6 g, 0.57 moles) was diluted with ethanol (80 ml) and added to the above slurry slowly over a period of 30 min at 22-30° C. The product slurry was further stirred at 22-30° C. for 4 hours. Further, it was cooled to ˜5° C. and stirred at this temperature for 60 min. The product was filtered and washed with precooled ethanol (60 ml). Product was dried under reduced pressure at 40±2° C. to obtain Lamivudine Form I.

Yield: 49.8 g

### EXAMPLE 18

### 4-amino-1-[(2R,5S)-2-(hydroxymethyl-1,3-Oxathiolan-5-yl)-(1H)-pyrimidin-2-one (Lamivudine Form I)

Lamivudine succinate monohydrate (100 g, 0.27 moles) was added to a mixture of methanol (230 ml) and water (30 ml) and the resulting slurry was stirred at 22-30° C. for ˜15 min. Triethylamine (57.6 g, 0.57 moles) was diluted with methanol (40 ml) and added to the above slurry slowly over a period of 30 min at 22-30° C. The product slurry was further stirred at 22-30° C. for 4 hours. Further, it was cooled to ˜5° C. and stirred at this temperature for 60 min. The product was filtered and washed with a mixture of methanol (40 ml) and ethyl acetate (60 ml). Product was dried under reduced pressure at 40±2° C. to obtain Lamivudine Form I.

Yield: 51.6 g

Methanol content: ˜1.3%

### EXAMPLE 19

### 4-amino-1-[(2R,5S)-2-(hydroxymethyl-1,3-oxathiolan-5-yl)-(1H)-pyrimidin-2-one (Lamivudine Form I)

Lamivudine succinate monohydrate (51 g, 0.14 moles) was suspended in a mixture of isopropanol (250 ml) and water (7.5 ml) at 23-28° C. Triethylamine (29.6 g, 0.29 moles) was added to the above suspension at 23-28° C. in about 20 min and the resulting slurry was stirred for 3 h at 23-28° C. The product slurry was cooled to 0-5° C. and stirred for 60 min. The product was filtered and washed with precooled isopropanol (100 ml, 5° C.). Dried the wet product under reduced pressure at ˜45° C. to obtain Lamivudine polymorph Form-I.

Yield: 28.5 g

SOR[α]_{D}²⁵ (c=1 in methanol on anhydrous basis): −142°

Water content: 1.7% w/w.

### EXAMPLE 20

### 4-amino-1-≡(2R,5S)-2-(hydroxymethyl-1,3-oxathiolan-5-yl)-(1H)-pyrimidin-2-one (Lamivudine Form I)

Lamivudine succinate monohydrate (51 g, 0.14 moles, pulverized) was suspended in a mixture of isopropanol (330 ml) and water (11 ml) at 23-28° C. Triethylamine (30 g, 0.30 moles) was diluted with isopropanol, (40 ml) and added to the above suspension at 23-28° C. in about 20 min and the resulting slurry was stirred for 3 h at 23-28° C. The product slurry was cooled to 8-12° C. and stirred for 60 min. The product was filtered and washed with precooled aqueous isopropanol (Mixture of 60 ml isopropanol and 1.2 ml water, chilled to ˜5° C.). Dried the wet product under reduced pressure at ˜45° C. till water content was between 1.5-2.0% w/w (by KF). The product obtained was Lamivudine polymorph Form-I (with Lamivudine polymorph Form-II below the limit of quantification by Raman Spectroscopic analysis).

Yield: 28.5 g

SOR[α]_{D}²⁵ (c=1 in methanol on anhydrous basis): −142°

Water content: 1.7% w/w.

IPA content: 0.12% w/w (1204 ppm)

### EXAMPLE 21

### 4-amino-1-[(2R,5S)-2-(hydroxymethyl-1,3-oxathiolan-5-yl)-(1H)-pyrimidin-2-one (Lamivudine Form I)

Lamivudine succinate monohydrate (73 g, 0.2 moles) was dissolved in a mixture of isopropanol (475 ml) and water (25 ml) at 58-63° C. The solution was treated with carbon (4 g) and filtered through a hyflo bed in hot condition to get a clear filtrate. The residue was washed with preheated (˜65° C.) mixture of isopropanol (47.5 ml) and water (2.5 ml). Filtrate and washing was combined and concentrated under reduced pressure at 45-50° C. to obtain a residue. To the residue was added isopropanol (450 ml) and stirred to get a uniform slurry. The moisture content of the slurry was adjusted to 4.3-4.8% w/w (˜4.5% w/w) by adding water. Triethylamine (43 g) was diluted with isopropanol (60 ml) and added slowly in 30-40 min to the slurry at 23-28° C. The resulting slurry was stirred for 4 h at 23-28° C. The product slurry was cooled to 5-8° C. and stirred for 60 min. The product was filtered and washed with precooled aqueous isopropanol (Mixture of 80 ml isopropanol and 1.6 ml water, chilled to ˜5° C.). Wet product is dried under reduced pressure at 45-50° C. till water content was between 1.5-2.0% w/w (by KF). The product obtained was Lamivudine polymorph Form-I (with no contamination of Lamivudine polymorphic Form-II, by Raman/XRD/IR analysis).

Yield: 40.2 g

SOR[α]_{D}²⁵ (c=1 in methanol on anhydrous basis): −142.2°

Water content: 1.66% w/w.

IPA content: 0.12% w/w (1200 ppm)

### EXAMPLE 22

### 4-amino-1-[(2R,5S)-2-(hydroxymethyl-1,3-oxathiolan-5-yl)-(1H)-pyrimidin-2-ONE (Lamivudine Form I)

Lamivudine succinate monohydrate (100 g, 0.27 moles) was added to a mixture of ethanol (210 ml) and water (24 ml) and the resulting slurry was stirred at 22-30° C. for ˜15 min. Triethylamine (57.6 g, 0.57 moles) was diluted with ethanol (80 ml) and added to the above slurry slowly over a period of 30 min at 22-30° C. The product slurry was further stirred at 22-30° C. for 4 hours. Further, it was cooled to ˜5° C. and stirred at this temperature for 60 min. The product was filtered and washed with precooled ethanol (60 ml). Product was dried under reduced pressure at 40±2° C. to obtain Lamivudine Form I.

Yield: 49.8 g

### EXAMPLE 23

### 4-amino-1-[(2R,5S)-2-(hydroxymethyl-1,3-oxathiolan-5-yl)-(1H)-pyrimidin-2-one (Lamivudine Form I)

Lamivudine succinate monohydrate (100 g, 0.27 moles) was added to a mixture of methanol (230 ml) and water (30 ml) and the resulting slurry was stirred at 22-30° C. for ˜15 min, Triethylamine (57.6 g, 0.57 moles) was diluted with methanol (40 ml) and added to the above slurry slowly over a period of 30 min at 22-30° C. The product slurry was further stirred at 22-30° C. for 4 hours. Further, it was cooled to ˜5° C. and stirred at this temperature for 60 min. The product was filtered and washed with a mixture of methanol (40 ml) and ethyl acetate (60 ml). Product was dried under reduced pressure at 40±2° C. to obtain Lamivudine Form I.

Yield: 51.6 g

Methanol content: ˜1.3%

### EXAMPLE 24

### 4-amino-1-[(2R,5S)-2-(hydroxymethyl-1,3-oxathiolan-5-yl)-(1H)-pyrimidin-2-one (Lamivudine Polymorphic form-II)

Lamivudine succinate monohydrate (100 g, 0.27 moles) was added to ethanol (675 ml) and the resulting slurry was heated to 65-70° C. to obtain a clear solution. The solution was treated with carbon (4 g) at 65-70° C. for 10-15 min and filtered through hytlo in hot condition and the residue was washed with preheated ethanol (90 ml, 50-55° C.) to obtain a clear filtrate. To the clear filtrate triethylamine (60.3 g, 0.60 moles) was added slowly in 10-15 min at 47-55° C. Ethanol (˜110 ml) was distilled under atmospheric pressure at ˜83° C. Further, ethanol (˜170 ml) was distilled under reduced pressure at 50-60° C. to achieve reaction mass volume ˜550 ml. Lamivudine Form-II seed (0.1 g) is added to the concentrated mass at ˜50° C. and it is cooled to 22-30° C. Stirring is continued at 22-30° C. for 2 h. Finally, the product slurry is cooled to 10-13° C. and continued stirring at this temperature for 3 h. The product was filtered and washed with precooled ethanol (50 ml, 0-5° C.) containing triethylamine (0.50 g). Further, product is washed with a precooled mixture of ethanol and Ethylacetate (100 ml, 12-15° C. prepared by mixing 40 ml ethanol with 60 ml ethyl acetate). Product was dried under reduced pressure at 50-55° C. to obtain Lamivudine Form-II (by IR, XRD, Raman analysis).

Yield: 51.2 g

### EXAMPLE 25

### 4-amino-1-[(2R,5S)-2-(hydroxymethyl-1,3-oxathiolan-5-yl)-(1H)-pyrimidin-2-one (Lamivudine Polymorphic Form-II)

Lamivudine succinate monohydrate (100 g, 0.27 moles) was added to ethanol (800 ml) and the resulting slurry was heated to 47-49° C. Triethylamine (57.8 g, 0.57 moles) was added to the above slurry slowly over a period of 20 min at 47-52° C. The product slurry was further stirred at 47-52° C. for 20 min. The product slurry was further heated to reflux (˜80° C.) to get clear solution. Initially ethanol (140-150 ml) is distilled at atmospheric pressure at ˜83° C. and further ethanol (˜390 ml) is recovered under reduced pressure (100-150 mm Hg) at 55-60° C. To the concentrate ethyl acetate (75 ml) is added over a period of 10-15 min at 55-60° C. and seeded with Lamivudine Form-II (0.1 g). Stirring is continued at ˜55° C. for 15-20 min to initiate the crystallization. Further, the reaction mass is diluted with a mixture of ethyl acetate (500 ml) and triethylamine (0.75 g) at ˜55° C. Product slurry is cooled to 23-28° C. and stirring was continued at 23-28° C. for 4 h. The product was filtered and washed with ethyl acetate (100 ml). Product was dried under reduced pressure at 50-55° C. to obtain Lamivudine Form-II.

Yield: 52.8 g

### EXAMPLE 26

### 4-amino-1-[(2R,5S)-2-(hydroxymethyl-1,3-oxathiolan-5-yl)-(1H)-pyrimidin-2-one (Lamivudine Polymorphic Form-II)

Lamivudine succinate monohydrate (100 g, 0.27 moles) was added to ethanol (300 ml) and the resulting slurry was stirred at 22-30° C. for ˜15 min. Triethylamine (57.6 g, 0.57 moles) was diluted with ethanol (50 ml) and added to the above slurry slowly over a period of 30 min at 22-30° C. Product slurry was stirred at 22-30° C. for 4 h. The product was filtered and washed with precooled ethanol (50 ml). Product was dried under reduced pressure at 50-55° C. to obtain Lamivudine Form-II.

Yield: 45 g

We claim:

1) An improved process to prepare Lamivudine polymorphic Form I, by treating Lamivudine salt selected from succinate salt or cinnamate salt in an organic solvent or in a mixture of organic solvent and water in the presence of a base.

2) (canceled)

3) The process according to claim 1, wherein the organic solvent is selected from ethyl acetate, methyl acetate, ethanol, methanol, isopropyl alcohol, n-propanol, n-butanol, acetone or mixtures thereof.

4) The process according to claim 1, wherein the base is selected from triethyl amine, ammonium hydroxide, ammonia or t-butyl amine.

5) An improved process to prepare Lamivudine polymorphic Form II, by treating Lamivudine salt selected from succinate salt or cinnamate salt in an organic solvent in the presence of a base.

6) The process according to claim 5, wherein the organic solvent is selected from ethanol, methanol or isopropyl alcohol.

7) The process according to claim 5, wherein the base is selected from triethyl amine, diethyl amine or diisopropyl amine.

8) An improved process to prepare a cis-nucleoside derivative of Formula I, 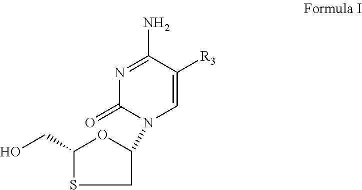
wherein R₃ represents H, F, Cl, C₁₋₁₆ alkyl.
which comprises:
a) reacting 1,3-oxathiolane compound of Formula IV, 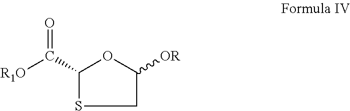
wherein R represents acyl group selected from —COCH₃, —COC₂H₅, —COCH₂Cl, —COCH₂Br, —COC₆H₅, —COR_{S}; R₅ represents substituted phenyl group selected from 4-nitrophenyl, 4-chlorophenyl; R₁ represents hydrogen, alkyl, aralkyl, alkenyl, aryl preferably an alkyl group/substituted alkyl group, more preferably a chiral auxiliary with one or more chiral centres such as d(+)menthol or l(−)menthol with a pyrimidine base of Formula V, 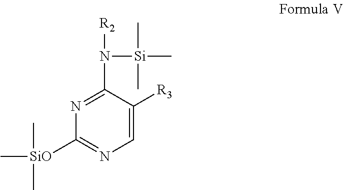
wherein R₂ represents COR₄; R₄ represents H, C₁₋₆ alkyl; R₃ represents H, F, Cl, C₁₋₁₆ alkyl,
in the presence of trityl glycosidation agent to give protected nucleoside of [2R, 5S] compound of Formula VI and [2R, 5R] compound of Formula VII in proportion of 70:30 to 80:20; 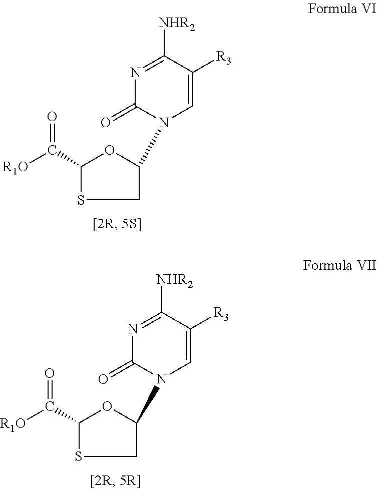
wherein R₁, R₂ and R₃ are same as defined above.
b) optionally isolating the compound of Formula VI;
c) deprotecting the mixture of [2R, 5S] compound of Formula VI and [2R, 5R] compound of Formula VII in presence of an acid to form a [2R, 5S] compound of Formula VIII and [2R, 5R] compound of Formula IX; 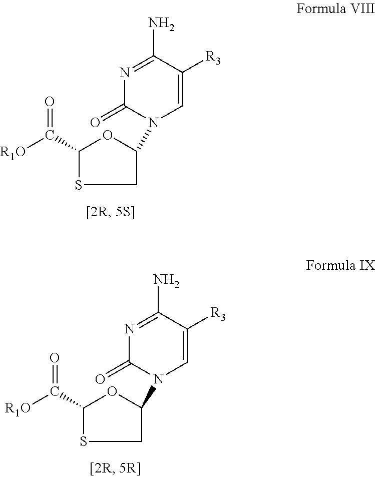
wherein R₁, and R₃ are same as defined above.
d) isolating the [2R, 5S] compound of Formula VIII;
e) reducing the [2R, 5S] cis nucleoside compound of Formula VIII to give a compound of Formula I; and
f) isolating the cis-nucleoside derivative of formula I.

9) The process according to claim 8, wherein the trityl glycosidation agent in step (a) is selected from trityl perchlorate of compound of Formula X, 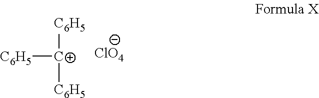
or trityl fluoroborate of compound of Formula XI,

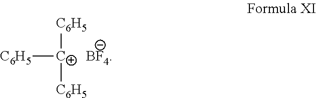

10) The process according to claim 8, wherein trityl glycosidation agent is used in the range of 0.3-1.3 equivalent moles based on the 1,3-oxathiolane compound of Formula IV.

11) (canceled)

12) The process according to claim 8, wherein the step (a) reaction is carried out in a solvent selected from halogenated hydrocarbons such as methylene chloride, ethylene chloride; hydrocarbon such as toluene; a nitrile such as acetonitrile; an ether such as tetrahydrofuran; 1,2-dimethoxy ethane (DME) or mixtures thereof.

13) The process according to claim 8, wherein the deprotection of the compound of Formula VI is carried out using an acid selected from trifluoroacetic acid, methane sulfonic acid, p-toluenesulfonic acid in an alcoholic solvent preferably in absolute ethanol.

14) The process according to claim 13, wherein the deprotected cis nucleoside compound of Formula VIII and compound of Formula IX in step (c) is isolated in the form of acid addition salt as such from the reaction mass or by adding an anti solvent selected from hydrocarbons selected from toluene, hexanes, cyclohexane or an ether solvent selected from diisopropylether or an ester solvent selected from ethylacetate, isopropylacetate to give compound of Formula XVII and compound of Formula XVIII, 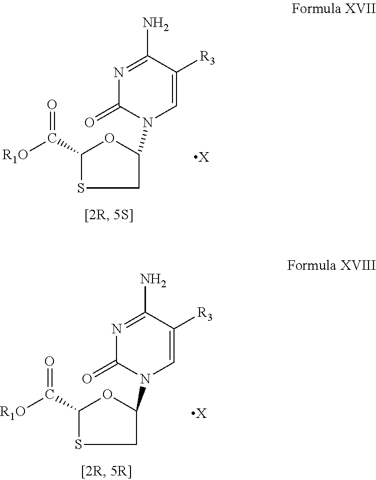
wherein R₁, and R₃ are same as defined above.

15) The process according to claim 8, wherein step (d) the deprotected nucleoside of compound of Formula VIII is isolated by adding an organic base selected from triethylamine in a mixture of solvents selected from a mixture of ethylacetate, hexanes and water.

16) The process according to claim 8, wherein the reduction in step (e), is carried out using sodium borohydride in aqueous alcoholic solvent, mixture of alcoholic solvent with water or aqueous tetrahydrofuran.

17) The process according to claim 8, wherein after the reduction the cis-nucleoside is isolated as a succinate salt or cinnamate salt.

18) The process according to claim 8, wherein the succinate salt or cinnamate salt of cis-nucleoside is hydrolysed to give cis-nucleoside of compound of Formula I.

19) The process according to claim 8, wherein the cis-nucleoside derivative is isolated from aqueous solution as water insoluble sulfoxide, by adding an oxidizing agent selected from hydrogen peroxide.

20) The process according to claim 19, wherein the sulfoxide formed is treated with phosphorus pentasulfide (P₄S₁₀) in an organic solvent selected from methylene chloride, toluene, acetone, pyridine, carbon disulphide or mixture thereof to carry out deoxygenation and to obtain cis-nucleoside of Formula I.

21) The process according to claim 8, wherein the cis-nucleoside of Formula I is selected from Lamivudinen or Emtricitabine.

22) The process according to claim 8, wherein the Lamivudine is isolated as polymorphic Form I.

23) The process according to claim 8, wherein the Lamivudine is isolated as polymorphic Form II.

24) The compound of Formula XII,

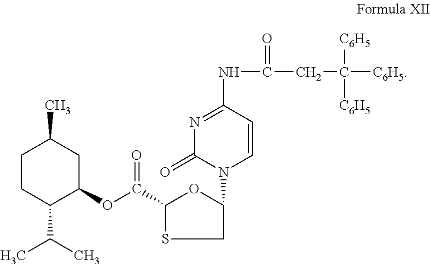

25) The compound of Formula XIII, 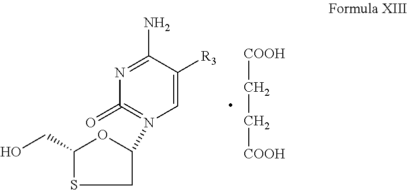
wherein R₃ represents H, F, Cl, C₁₋₁₆ alkyl.

26) (canceled)

27) The compound of Formula XIV, 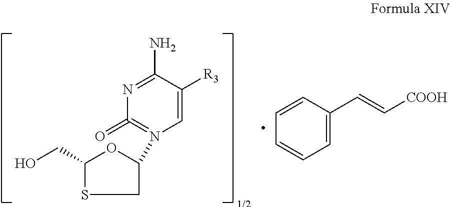
wherein R₃ represents H, F, Cl, C₁₋₁₆ alkyl.

28) The compound of Formula XVI, 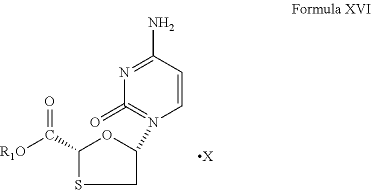
wherein R₁ represents hydrogen, alkyl, aralkyl, alkenyl, aryl preferably an alkyl group/substituted alkyl group, more preferably a chiral auxiliary with one or more chiral centres such as d(+)menthol or l(−)menthol; X represents methanesulphonic acid, trifluoroacetic acid, p-toluenesulfonic acid.
